(19) United States
(12) Patent Application Publication
(10) Pub. No.: US 2023/0103184 A1
(43) Pub. Date: Mar. 30, 2023

(54) NASAL ASPIRATOR WITH FINGER GRIPS

(71) Applicant: George Pollock, Lake Wales, FL (US)

(72) Inventor: George Pollock, Lake Wales, FL (US)

(21) Appl. No.: 17/484,558

(22) Filed: Sep. 24, 2021

(51) Int. Cl. A61M 1/00

(52) U.S. Cl. CPC A61M 1/682; A61M 2205/586; A61M 2210/0618

(57) ABSTRACT

Disclosed are nasal aspirators with finger grips.

### FIELD

Some implementations relate generally to nasal aspirators, and, more particularly, to nasal aspirators with finger grips.

### BACKGROUND

Nasal aspirators are medical devices that are used to aspirate nasal passages. For example, nasal aspirators are often used during the birth process to aspirate the nasal passages or nostrils of a newborn. In such environments and others, there may be fluids on the patients or health care providers, which can make gripping a nasal aspirator difficult. Further, if a nasal aspirator slips from the grip of a health care provider and falls onto the floor, the nasal aspirator may have to be discarded and a new nasal aspirator obtained. This may result in delays in treating a patient and an increase in costs for expended supplies.

Embodiments were conceived in light of the above-mentioned problems and limitations, among other things. The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

### SUMMARY

Some implementations can include a nasal aspirator comprising a compressible bulb at a proximate end, an elongate nozzle at a distal end, and a set of annular finger grips disposed near a transition from the compressible bulb to the elongate nozzle, wherein the set of annular finger grips include a first annular finger grip and a second annular finger grip disposed opposite the first finger grip.

Some implementations can include a nasal aspirator comprising a compressible bulb at a proximate end, an elongate nozzle at a distal end, and a set of arcuate finger grips disposed near a transition from the compressible bulb to the elongate nozzle, wherein the set of arcuate finger grips include a first acuate finger grip and a second arcuate finger grip disposed opposite the first finger grip.

Some implementations can include a nasal aspirator comprising a compressible bulb at a proximate end, an elongate nozzle at a distal end, and a removable finger grip member including a central aperture configured to slide over the distal end of the elongate nozzle and to be secured, via an interference fit, at a transition from the compressible bulb to the elongate nozzle, wherein the removable finger grip member includes a first finger grip on a first side of the aperture and a second finger grip disposed opposite the first finger grip on a second side of the aperture.

### BRIEF DESCRIPTION OF THE DRAWINGS

FIG. 1 is a front elevation view of a first example nasal aspirator with finger grips in accordance with some implementations.

FIG. 2 is a rear elevation view of the first example nasal aspirator with finger grips in accordance with some implementations.

FIG. 3 is a side elevation view of the first example nasal aspirator with finger grips in accordance with some implementations.

FIG. 4 is a top elevation view of the first example nasal aspirator with finger grips in accordance with some implementations.

FIG. 5 is a bottom elevation of the first example nasal aspirator with finger grips in accordance with some implementations.

FIGS. 6-8 show the first example nasal aspirator with finger grips in use in accordance with some implementations.

FIG. 9 is a front elevation view of a second example nasal aspirator with finger grips in accordance with some implementations.

FIG. 10 is a rear elevation view of the second example nasal aspirator with finger grips in accordance with some implementations.

FIG. 11 is a side elevation view of the second example nasal aspirator with finger grips in accordance with some implementations.

FIG. 12 is a top elevation view of the second example nasal aspirator with finger grips in accordance with some implementations.

FIG. 13 is a bottom elevation of the second example nasal aspirator with finger grips in accordance with some implementations.

FIGS. 14-16 show the second example nasal aspirator with finger grips in use in accordance with some implementations.

FIG. 17 is a front elevation view of a third example nasal aspirator with finger grips in accordance with some implementations.

FIG. 18 is a rear elevation view of the third example nasal aspirator with finger grips in accordance with some implementations.

FIG. 19 is a side elevation view of the third example nasal aspirator with finger grips in accordance with some implementations.

FIG. 20 is a top elevation view of the third example nasal aspirator with finger grips in accordance with some implementations.

FIG. 21 is a bottom elevation of the third example nasal aspirator with finger grips in accordance with some implementations.

FIGS. 22-24 show the third example nasal aspirator with finger grips in use in accordance with some implementations.

### DETAILED DESCRIPTION

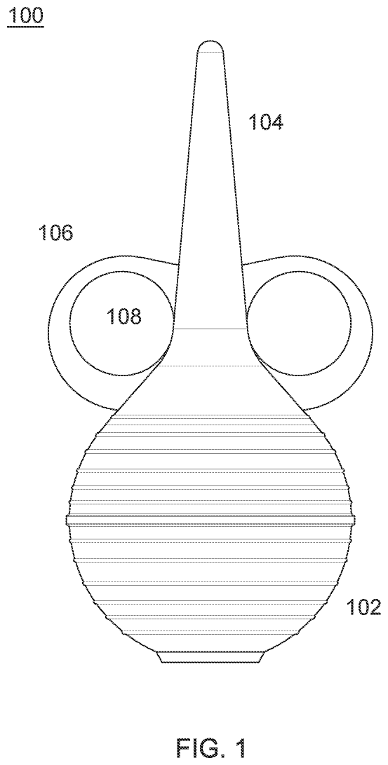

FIG. 1 is a front elevation view of a first example nasal aspirator 100 with a compressible bulb 102 at a proximate end, an elongate nozzle 104 at a distal end, and a set of annular finger grips 106 disposed near a transition from the compressible bulb to the elongate nozzle, wherein the set of annular finger grips include a first annular finger grip 108 and a second annular finger grip 110 disposed opposite the first finger grip.

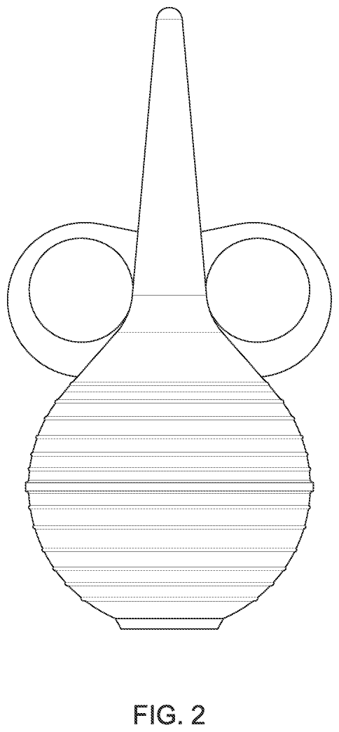

FIG. 2 is a rear elevation view of the first example nasal aspirator with finger grips in accordance with some implementations.

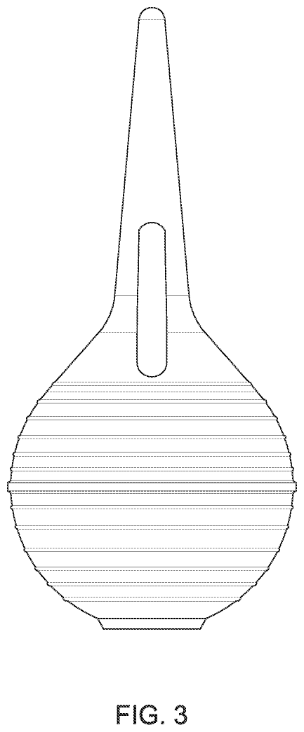

FIG. 3 is a side elevation view of the first example nasal aspirator with finger grips in accordance with some implementations.

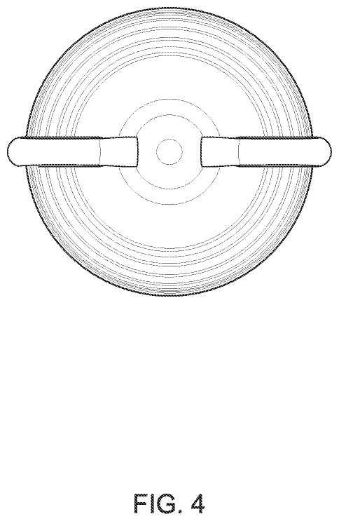

FIG. 4 is a top elevation view of the first example nasal aspirator with finger grips in accordance with some implementations.

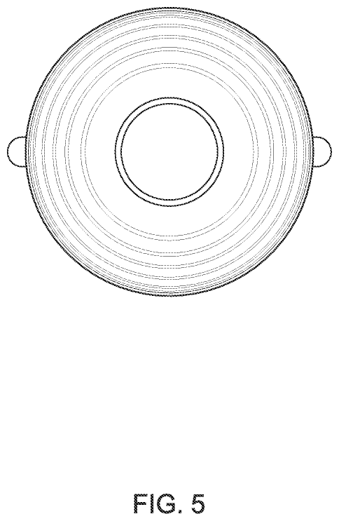

FIG. 5 is a bottom elevation of the first example nasal aspirator with finger grips in accordance with some implementations.

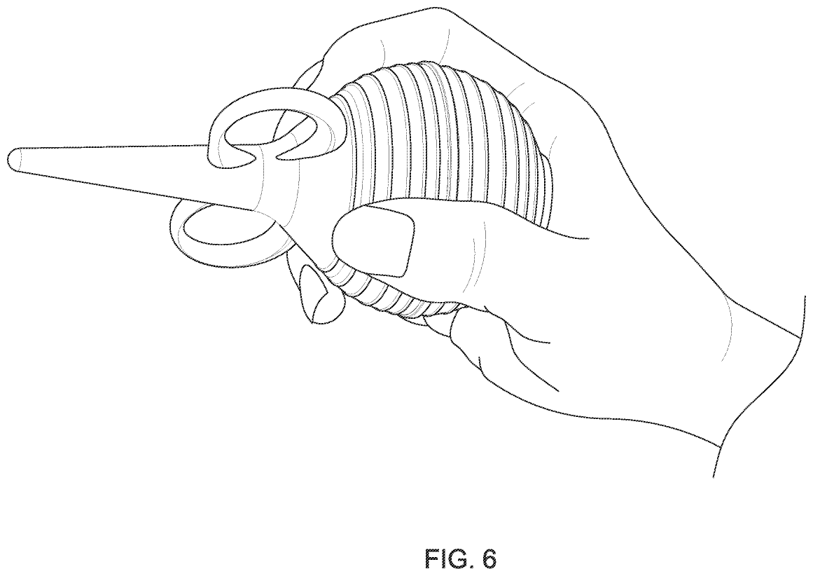

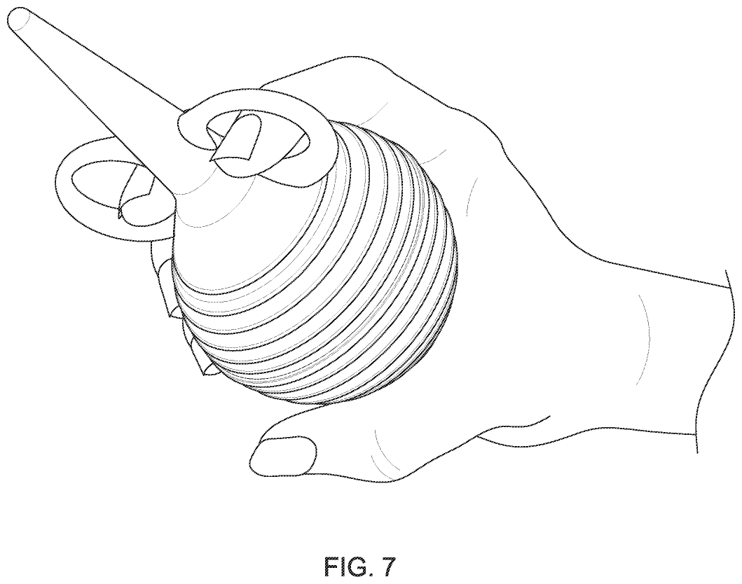

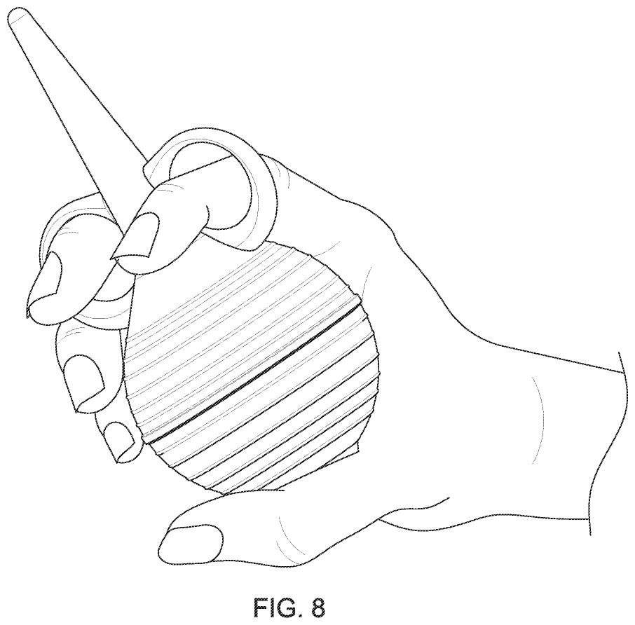

FIGS. 6-8 show the first example nasal aspirator with finger grips in use in accordance with some implementations. In operation, a user can insert a finger in each of the annular finger grips to help the user to keep a grip on the nasal aspirator and help prevent the nasal aspirator from slipping from the grip of the user.

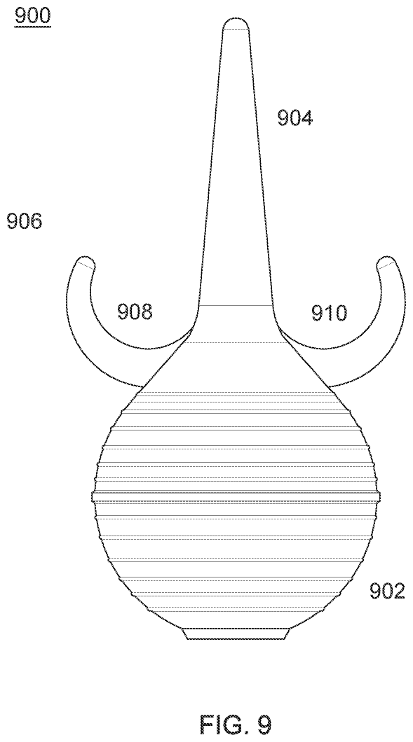

FIG. 9 is a front elevation view of a second example nasal aspirator 900 with a compressible bulb 902 at a proximate end, an elongate nozzle 904 at a distal end, a set of arcuate finger grips 906 disposed near a transition from the compressible bulb to the elongate nozzle, wherein the set of arcuate finger grips 906 include a first acuate finger grip 908 and a second arcuate finger grip 910 disposed opposite the first finger grip 908.

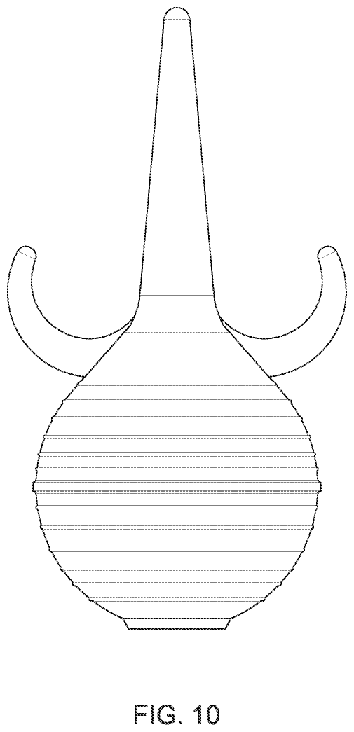

FIG. 10 is a rear elevation view of the second example nasal aspirator with finger grips in accordance with some implementations.

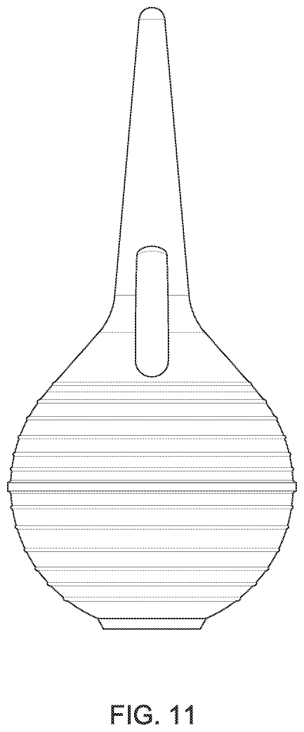

FIG. 11 is a side elevation view of the second example nasal aspirator with finger grips in accordance with some implementations.

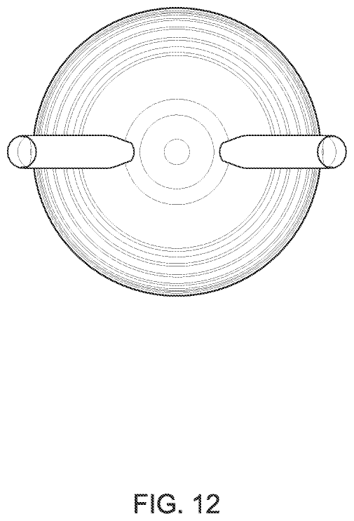

FIG. 12 is a top elevation view of the second example nasal aspirator with finger grips in accordance with some implementations.

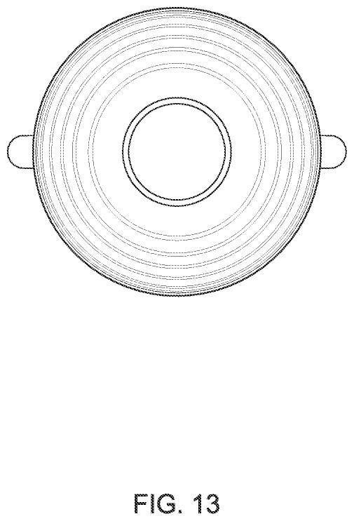

FIG. 13 is a bottom elevation of the second example nasal aspirator with finger grips in accordance with some implementations.

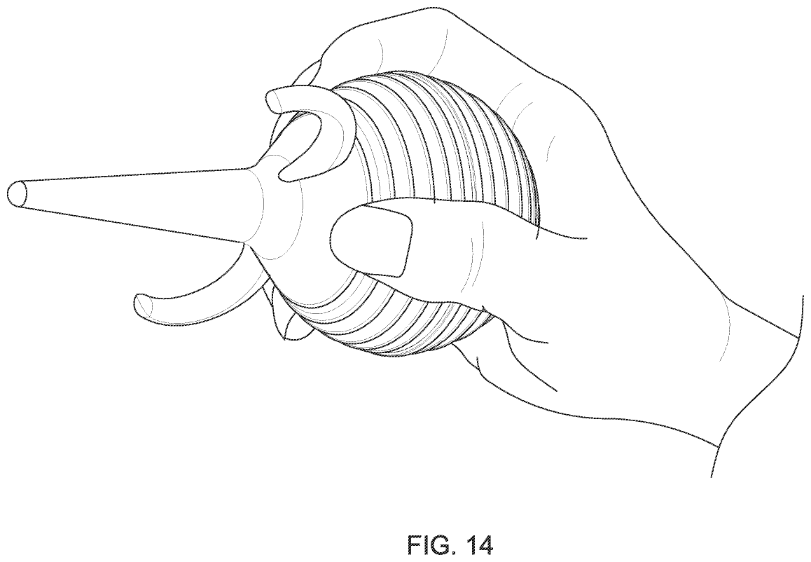

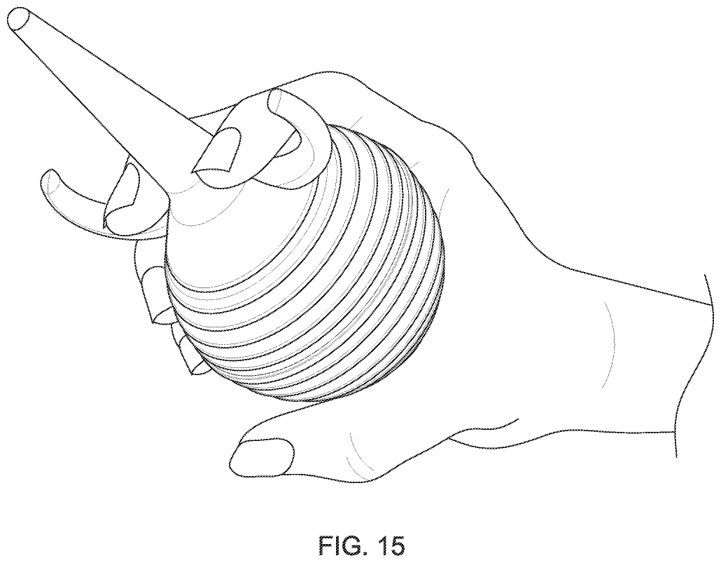

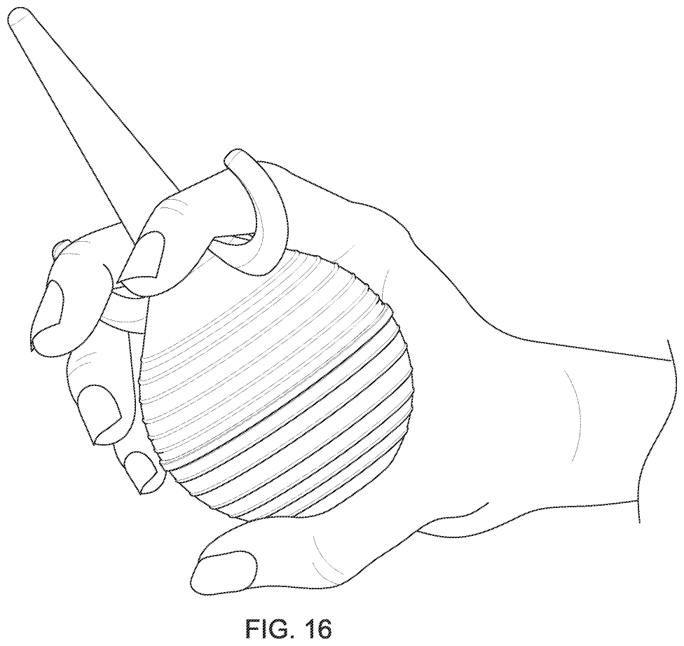

FIGS. 14-16 show the second example nasal aspirator with finger grips in use in accordance with some implementations. In operation, a user can insert a finger in each of the arcuate finger grips to help the user to keep a grip on the nasal aspirator and help prevent the nasal aspirator from slipping from the grip of the user.

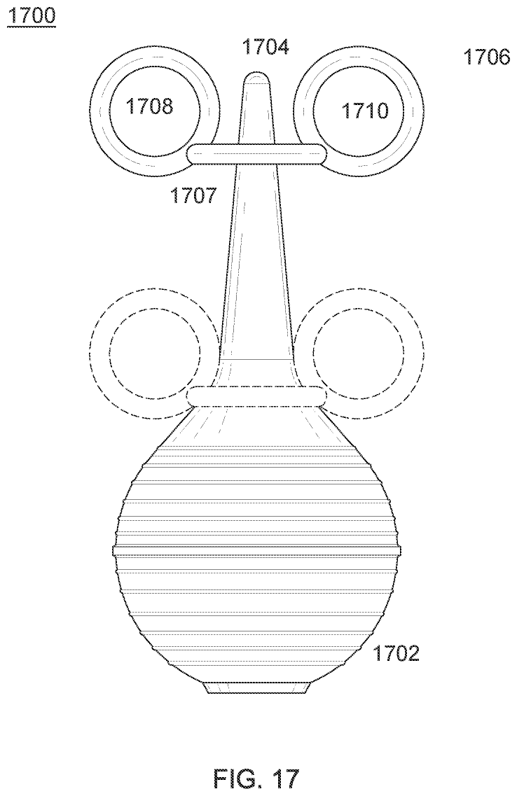

FIG. 17 is a front elevation view of a third example nasal aspirator 1700 comprising a compressible bulb 1702 at a proximate end, an elongate nozzle 1704 at a distal end, and a removable finger grip member 1706 including a central aperture 1707 configured to slide over the distal end of the elongate nozzle and to be secured, via an interference fit, at a transition from the compressible bulb to the elongate nozzle, wherein the removable finger grip 1706 member includes a first finger grip 1708 on a first side of the aperture and a second finger grip 1710 disposed opposite the first finger grip 1708 on a second side of the aperture.

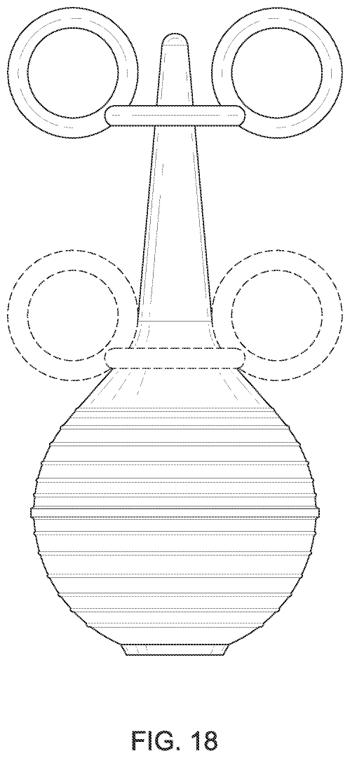

FIG. 18 is a rear elevation view of the third example nasal aspirator with finger grips in accordance with some implementations.

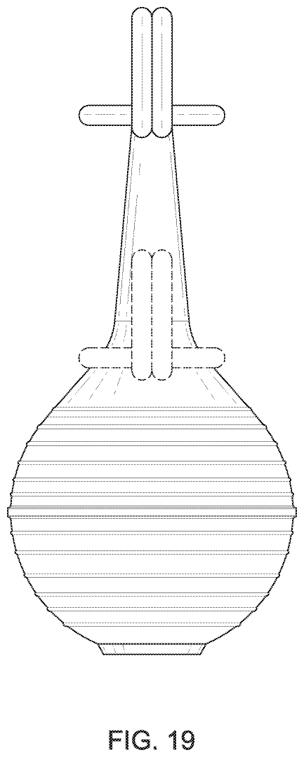

FIG. 19 is a side elevation view of the third example nasal aspirator with finger grips in accordance with some implementations.

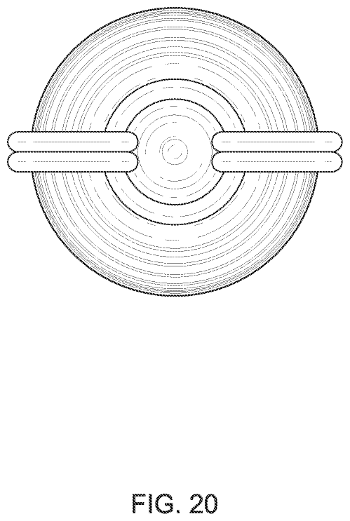

FIG. 20 is a top elevation view of the third example nasal aspirator with finger grips in accordance with some implementations.

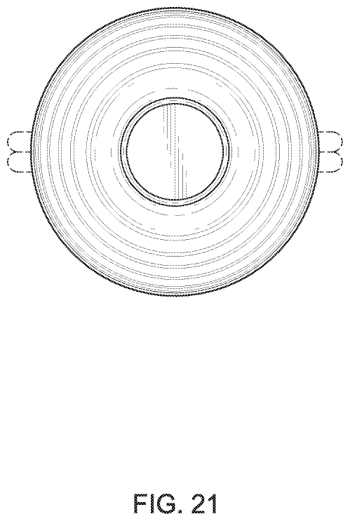

FIG. 21 is a bottom elevation of the third example nasal aspirator with finger grips in accordance with some implementations.

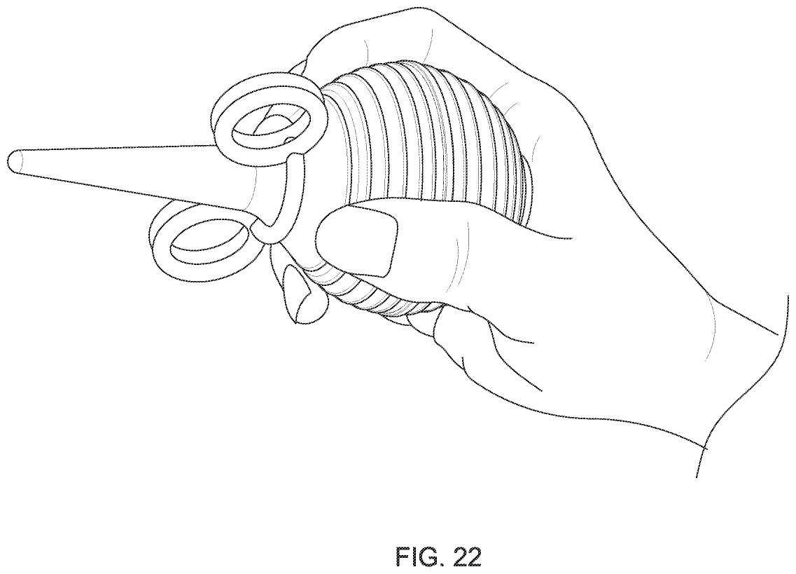

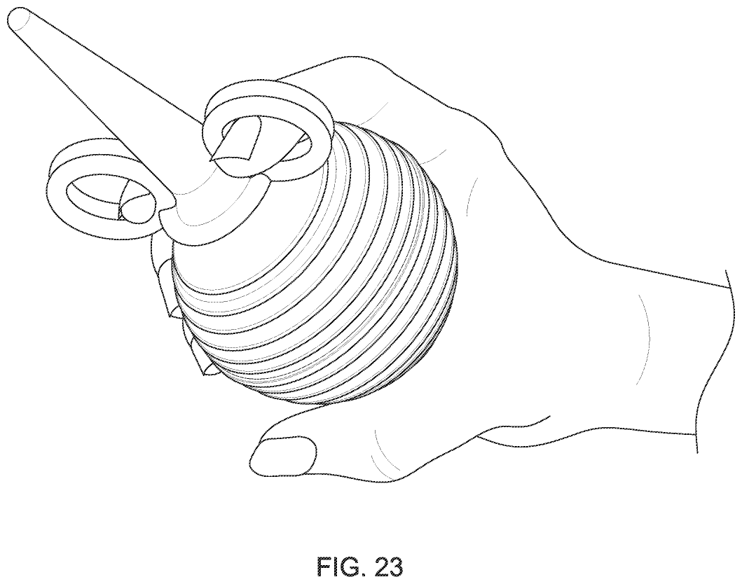

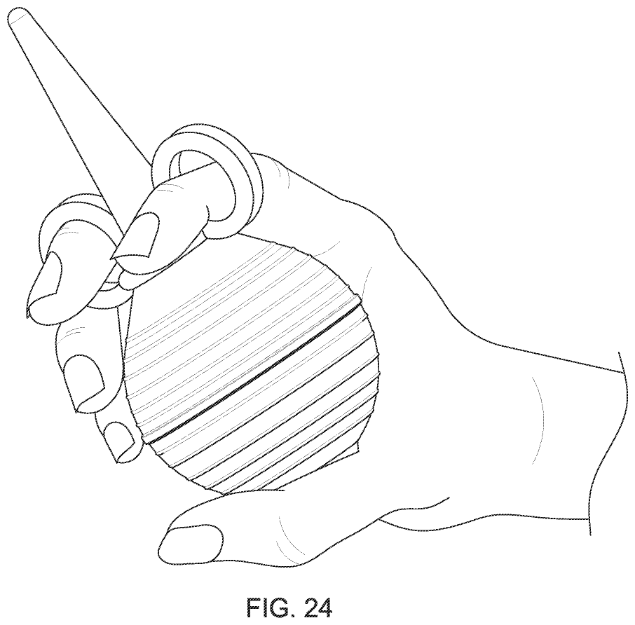

FIGS. 22-24 show the third example nasal aspirator with finger grips in use in accordance with some implementations. In operation, a user can insert a finger in each of the finger grips to help the user to keep a grip on the nasal aspirator and help prevent the nasal aspirator from slipping from the grip of the user.

While some example implementations have been described in terms of a general embodiment with several specific example modifications, it is recognized that other modifications and variations of the embodiments described above are within the spirit and scope of the disclosed subject matter. Applicant intends to embrace any and all such modifications, variations and embodiments.

What is claimed is:

1. A nasal aspirator comprising:
a compressible bulb at a proximate end;
an elongate nozzle at a distal end; and
a set of annular finger grips disposed near a transition from the compressible bulb to the elongate nozzle, wherein the set of annular finger grips include a first annular finger grip and a second annular finger grip disposed opposite the first annular finger grip.

2. A nasal aspirator comprising:
a compressible bulb at a proximate end;
an elongate nozzle at a distal end; and
a set of arcuate finger grips disposed near a transition from the compressible bulb to the elongate nozzle, wherein the set of arcuate finger grips include a first acuate finger grip and a second arcuate finger grip disposed opposite the first arcuate finger grip.

3. A nasal aspirator comprising:
a compressible bulb at a proximate end;
an elongate nozzle at a distal end; and
a removable finger grip member including a central aperture configured to slide over the distal end of the elongate nozzle and to be secured, via an interference fit, at a transition from the compressible bulb to the elongate nozzle, wherein the removable finger grip member includes a first finger grip on a first side of the aperture and a second finger grip disposed opposite the first finger grip on a second side of the aperture.
